(19) United States
(12) Patent Application Publication
(10) Pub. No.: US 2025/0161175 A1
(43) Pub. Date: May 22, 2025

(54) Oral Care Compositions

(71) Applicant: Colgate-Palmolive Company, New York, NY (US)

(72) Inventors: Rong DONG, Highland Park, NJ (US); Gregory SZEWCZYK, Flemington, NJ (US); Suman Kamar CHOPRA, Monroe, NJ (US); Santina Sally PERSICHETTI, Staten Island, NY (US)

(73) Assignee: Colgate-Palmolive Company, New York, NY (US)

(21) Appl. No.: 18/951,152

(22) Filed: Nov. 18, 2024

Related U.S. Application Data
(60) Provisional application No. 63/600,195, filed on Nov. 17, 2023.
(60) Provisional application No. 63/600,232, filed on Nov. 17, 2023.

(51) Int. Cl. A61K 8/22; A61K 8/34; A61K 8/81; A61Q 11/00

(52) U.S. Cl. CPC A61K 8/22; A61K 8/345; A61K 8/8147; A61Q 11/00; A61K 2800/28; A61K 2800/30

(57) ABSTRACT

Oral care compositions and, e.g., oral care compositions providing improved stain resistance are disclosed herein. In accordance with an aspect, provided is an oral care composition including a whitening agent comprising from about 0.1 to about 10 wt. % of a peroxide; a cross-linked polyacrylic acid and/or a salt thereof; a polyol; and water in an amount up to 10 wt. %, wherein all weight percentages are based on the total weight of the oral care composition.

### CROSS-REFERENCE TO RELATED APPLICATIONS

This application claims the benefit of priority from U.S. Patent Application No. 63/600,195, filed Nov. 17, 2023, titled Oral Care Compositions, and from U.S. Patent Application No. 63/600,232, filed Nov. 17, 2023, titled Oral Care Compositions, the content for both of which are hereby incorporated herein by reference in their entireties for all purposes.

### BACKGROUND

Oral care compositions, such as dentifrice compositions, can typically include one or more ingredients for maintaining the health of the oral cavity. Consumers have become aware of and desiring oral care compositions that also maintain the aesthetics of their teeth. It can be desirable to have dentifrice compositions with a relatively high level of peroxide of at least about 1 wt. % of the total weight of the oral care composition to achieve high whitening efficacy. However, it can be challenging to formulate dentifrice compositions with a relatively high level of peroxide because peroxide can be reactive with other dentifrice components, such as fluoride, metal ions, abrasives, etc.

Oral care compositions containing a relatively high level of peroxide have been particularly difficult to formulate with abrasives. One approach to the formulation challenges associated with incorporating sufficient peroxide in a dentifrice composition can be to separately package and/or apply a peroxide separate from the dentifrice composition. Unfortunately, consumer compliance can be lower when a peroxide must be applied separately from the dentifrice composition.

### BRIEF SUMMARY

This summary is intended merely to introduce a simplified summary of some aspects of one or more implementations of the present disclosure. Further areas of applicability of the present disclosure will become apparent from the detailed description provided hereinafter. This summary is not an extensive overview, nor is it intended to identify key or critical elements of the present teachings, nor to delineate the scope of the disclosure. Rather, its purpose is merely to present one or more concepts in simplified form as a prelude to the detailed description below.

Aspects of the invention are directed to oral care compositions and, e.g., oral care compositions providing improved stain resistance. In accordance with an aspect, provided is an oral care composition including a whitening agent comprising from about 0.1 to about 10 wt. % of a peroxide; a cross-linked polyacrylic acid and/or a salt thereof, a polyol; and water in an amount up to 10 wt. %, wherein all weight percentages are based on the total weight of the oral care composition.

In accordance with another aspect, provided is an oral care composition including a whitening agent comprising from 3 to about 8 wt. % of hydrogen peroxide; a carbomer; a polyol; and from about 1 to about 8 wt. % of water, wherein all weight percentages are based on the total weight of the oral care composition.

In accordance with a further aspect, provided is an oral care composition including a whitening agent comprising from 2 to about 10 wt. % of a peroxide; a cross-linked polyacrylic acid and/or a salt thereof, a polyol; and water in an amount up to 8 wt. %, wherein all weight percentages are based on the total weight of the oral care composition.

A list of non-limiting exemplary embodiments is provided below:

In accordance with an embodiment 1, provided is an oral care composition comprising: a whitening agent comprising from 0.1 to about 10 wt. % of a peroxide; a cross-linked polyacrylic acid and/or a salt thereof, a polyol; and water in an amount up to 10 wt. %, wherein all weight percentages are based on the total weight of the oral care composition.

In accordance with an embodiment 2, provided is the oral care composition according to embodiment 1, wherein the peroxide is present in an amount of about 3 to about 10 wt. %.

In accordance with an embodiment 3, provided is the oral care composition according to embodiment 1, wherein the peroxide is present in an amount of about 3.5 to 6 wt. %.

In accordance with an embodiment 4, provided is the oral care composition according to any foregoing embodiment, wherein the peroxide is selected from hydrogen peroxide, lithium peroxide, potassium peroxide, sodium peroxide, magnesium peroxide, calcium peroxide, barium peroxide, alkyl hydrogen peroxides, dialkyl peroxides, alkyl peroxy acids, peroxy esters, diacyl peroxides, benzoyl peroxide, monoperoxyphthalate, linear PVP complexed with hydrogen peroxide, cross-linked PVP complexed with hydrogen peroxide, silica complexed with hydrogen peroxide, tetrasodium pyrophosphate, complexed with hydrogen peroxide, and a combination of two or more thereof.

In accordance with an embodiment 5, provided is the oral care composition according to any foregoing embodiment, wherein the peroxide is a non-complexed peroxide.

In accordance with an embodiment 6, provided is the oral care composition according to any foregoing embodiment, wherein the peroxide is hydrogen peroxide.

In accordance with an embodiment 7, provided is the oral care composition according to any foregoing embodiment, wherein the cross-linked polyacrylic acid and/or a salt thereof is a branched polyacrylic acid and/or a salt thereof.

In accordance with an embodiment 8, provided is the oral care composition according to any foregoing embodiment, wherein the cross-linked polyacrylic acid and/or the salt thereof comprises a carbomer.

In accordance with an embodiment 9, provided is the oral care composition according to any foregoing embodiment, wherein the cross-linked polyacrylic acid and/or the salt thereof consist of one or more carbomer.

In accordance with an embodiment 10, provided is the oral care composition according to any foregoing embodiment, wherein the cross-linked polyacrylic acid and/or the salt thereof is present in an amount from about 0.2 to about 5 wt. %, optionally from about 0.2 to about 1 wt. %.

In accordance with an embodiment 11, provided is the oral care composition according to any foregoing embodiment, wherein the water is present in an amount from 1 to about 6 wt. %.

In accordance with an embodiment 12, provided is the oral care composition according to any foregoing embodiment further comprising: an abrasive, optionally, in an amount from about 1 to about 35 wt. %.

In accordance with an embodiment 13, provided is the oral care composition according to embodiment 12, wherein the abrasive comprises an insoluble phosphate salt.

In accordance with an embodiment 14, provided is the oral care composition according to embodiment 13, wherein the insoluble phosphate salt is selected from calcium pyrophosphate, dicalcium orthophosphate dihydrate, tricalcium phosphate, and calcium polymetaphosphate, and a combination of two or more thereof.

In accordance with an embodiment 15, provided is the oral care composition according to embodiment 12, wherein the abrasive consists of one or more calcium-based abrasive.

In accordance with an embodiment 16, provided is the oral care composition according to any foregoing embodiment, wherein the polyol is present in an amount from about 30 to about 70 wt. %.

In accordance with an embodiment 17, provided is the oral care composition according to any foregoing embodiment, wherein the polyol is selected from glycerin, sorbitol, ethylene glycol, propylene glycol, butylene glycol, pentylene glycol, hexylene glycol, diethylene glycol, triethylene glycol, tetraethylene glycol, pentaethylene glycol, dipropylene glycol, a polyethylene glycol, a polypropylene glycol, a block polymer of polyethylene glycol and polypropylene glycol, and a combination of two or more thereof.

In accordance with an embodiment 18, provided is the oral care composition according to any foregoing embodiment, wherein the polyol comprises a plurality of polyols.

In accordance with an embodiment 19, provided is the oral care composition according to embodiment 18, wherein at least one of the plurality of polyols is selected from a polyethylene glycol, a polypropylene glycol, a block polymer of polyethylene glycol and polypropylene glycol, and a combination of two or more thereof.

In accordance with an embodiment 20, provided is the oral care composition according to embodiment 18, wherein at least one of the plurality of polyols is selected from glycerin, ethylene glycol, propylene glycol, butylene glycol, pentylene glycol, hexylene glycol, dipropylene glycol, a polyethylene glycol, a polypropylene glycol, and a combination of two or more thereof.

In accordance with an embodiment 21, provided is the oral care composition according to any foregoing embodiment further comprising: a fluoride source, optionally, in an amount from about 0.1 to about 7 wt. %.

In accordance with an embodiment 22, provided is the oral care composition according to embodiment 21, wherein the fluoride source is selected from stannous fluoride, sodium fluoride, potassium fluoride, sodium monofluorophosphate, sodium fluorosilicate, ammonium fluorosilicate, amine fluoride, ammonium fluoride, and a combination of two or more thereof.

In accordance with an embodiment 23, provided is the oral care composition according to any foregoing embodiment further comprising: an anti-calculus agent in an amount from about 0.5 to about 10 wt. %.

In accordance with an embodiment 24, provided is the oral care composition according to embodiment 23, wherein the anti-calculus agent is selected from tetrasodium pyrophosphate, ammonium tripolyphosphate, hexametaphosphate salt, polycarboxylate polymers, polyvinyl methyl ether/maleic anhydride (PVME/MA) copolymers, and a combination of two or more thereof.

In accordance with an embodiment 25, provided is the oral care composition according to any foregoing embodiment, wherein the oral care composition is free of a peroxidase.

In accordance with an embodiment 26, provided is the oral care composition according to any foregoing embodiment, wherein the oral care composition is free of complexed peroxides.

In accordance with an embodiment 27, provided is an oral care composition comprising: a whitening agent comprising from 3 to about 8 wt. % of hydrogen peroxide; a carbomer; a polyol; and from about 1 to about 8 wt. % of water, wherein all weight percentages are based on the total weight of the oral care composition.

In accordance with an embodiment 28, provided is the oral care composition according to any foregoing embodiment, wherein the oral care composition has a relative dental abrasivity of about 10 to about 250, as determined pursuant to standards DIN EN ISO 11609.

### DETAILED DESCRIPTION

For illustrative purposes, the principles of the present invention are described by referencing various exemplary embodiments thereof. Although certain embodiments of the invention are specifically described herein, one of ordinary skill in the art will readily recognize that the same principles are equally applicable to, and can be employed in other compositions and methods. Before explaining the disclosed embodiments of the present invention in detail, it is to be understood that the invention is not limited in its application to the details of any particular embodiment disclosed herein. The terminology used herein is for the purpose of description and not of limitation.

As used herein and in the appended claims, the singular forms “a”, “an”, and “the” include plural references unless the context dictates otherwise. The singular form of any class of the ingredients refers not only to one chemical species within that class, but also to a mixture of those chemical species. The terms “a” (or “an”), “one or more” and “at least one” may be used interchangeably herein. The terms “comprising”, “including”, and “having” may be used interchangeably. The term “include” should be interpreted as “include, but are not limited to”. The term “including” should be interpreted as “including, but are not limited to”.

As used throughout, ranges are used as shorthand for describing each and every value that is within the range. Any value within the range can be selected as the terminus of the range. Thus, a range from 1-5, includes specifically 1, 2, 3, 4 and 5, as well as subranges such as 2-5, 3-5, 2-3, 2-4, 1-4, etc.

The term “about” when referring to a number means any number within a range of 10% of the number. For example, the phrase “about 2 wt. %” refers to a number between and including 1.8 wt. % and 2.2 wt. %.

All references cited herein are hereby incorporated by reference in their entireties. In the event of a conflict in a definition in the present disclosure and that of a cited reference, the present disclosure controls.

The abbreviations and symbols as used herein, unless indicated otherwise, take their ordinary meaning. The abbreviation “wt. %” means percent by weight with respect to the oral care composition. The symbol “°” refers to a degree, such as a temperature degree or a degree of an angle. The symbols “h”, “min”, “mL”, “nm”, and “pm” refer to hour, minute, milliliter, nanometer, and micrometer, respectively. The abbreviation “UV-VIS” referring to a spectrometer or spectroscopy, means Ultraviolet-Visible. The abbreviation “rpm” means revolutions per minute.

When referring to chemical structures, and names, the symbols “C”, “H”, and “O” mean carbon, hydrogen, and oxygen, respectively. The symbols “-”, “=” and “-” mean single bond, double bond, and triple bond, respectively.

“Volatile”, as used herein, means having a flash point of less than about 100° C. “Non-volatile”, as used herein, means having a flash point of greater than about 100° C.

Any member in a list of species that are used to exemplify or define a genus, may be mutually different from, or overlapping with, or a subset of, or equivalent to, or nearly the same as, or identical to, any other member of the list of species. Further, unless explicitly stated, such as when reciting a Markush group, the list of species that define or exemplify the genus is open, and it is given that other species may exist that define or exemplify the genus just as well as, or better than, any other species listed.

The phrases, “a mixture thereof,” “a combination thereof,” or a combination of two or more thereof” do not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.” Likewise, the term “a salt thereof” also relates to “salts thereof” Thus, where the disclosure refers to “an element selected from the group consisting of A, B, C, D, E, F, a salt thereof, and a mixture thereof,” it indicates that that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

All components and elements positively set forth in this disclosure can be negatively excluded from the claims. In other words, the oral care compositions of the instant disclosure can be free or essentially free of all components and elements positively recited throughout the instant disclosure. In some instances, the oral care compositions of the present disclosure may be substantially free of non-incidental amounts of the ingredient(s) or compound(s) described herein.

A non-incidental amount of an ingredient or compound is the amount of that ingredient or compound that is added into the oral care composition by itself. For example, an oral care composition may be substantially free of a non-incidental amount of an ingredient or compound, although such ingredient(s) or compound(s) may be present as part of a raw material that is included as a blend of two or more compounds.

Some of the various categories of components identified may overlap. In such cases where overlap may exist and the oral care composition includes both components (or the composition includes more than two components that overlap), an overlapping compound does not represent more than one component. For example, certain compounds may be characterized as both a polyol and a sweetener. If a particular oral care composition recites both a polyol and a sweetener, xylitol will serve only as either a polyol or a sweetener-not both.

For readability purposes, the chemical functional groups are in their adjective form; for each of the adjectives, the word “group” is assumed. For example, the adjective “alkyl” without a noun thereafter, should be read as “an alkyl group.”

Aspects of the invention are directed to oral care compositions and, e.g., oral care compositions providing improved stain resistance. In addition to achieving a whitening effect, it was unexpectedly discovered that oral care compositions according embodiments disclosed herein can achieve improved stain resistance. Thus, in certain embodiments, the oral care compositions provide both a reduction in the amount of present stain and provides improved resistance to future staining.

In accordance with an aspect of the invention, provided is an oral care composition including a whitening agent comprising from about 0.1 to about 10 wt. % of a peroxide; a cross-linked polyacrylic acid and/or a salt thereof; a polyol; and water in an amount up to 10 wt. %, wherein all weight percentages are based on the total weight of the oral care composition.

In accordance with another aspect of the invention, provided is an oral care composition including a whitening agent comprising from 3 to about 8 wt. % of hydrogen peroxide; a carbomer; a polyol; and from about 1 to about 8 wt. % of water, wherein all weight percentages are based on the total weight of the oral care composition.

In accordance with a further aspect, provided is an oral care composition including a whitening agent comprising from 2 to about 10 wt. % of a peroxide; a cross-linked polyacrylic acid and/or a salt thereof, a polyol; and water in an amount up to 8 wt. %, wherein all weight percentages are based on the total weight of the oral care composition.

The oral care compositions may preferably have a reduced amount, be substantially free, or free of peroxidase in some instances. For example, the oral care composition may preferably have about 4 wt. % or less, about 3 wt. % or less, about 2 wt. % or less, about 1 wt. % or less, about 0.5 wt. % or less, or about 0.1 wt. % or less of peroxidase, based on the weight of the oral care composition. In at least one embodiment, the oral care composition contains about 0 wt. % or 0 wt. % of peroxidase, based on the weight of the oral care composition.

Additionally or alternatively, the oral care compositions may preferably have a reduced amount, be substantially free, or free of complexed peroxides. In some preferred instances, the oral care compositions have about 4 wt. % or less, about 3 wt. % or less, about 2 wt. % or less, about 1 wt. % or less, about 0.5 wt. % or less, or about 0.1 wt. % or less of complexed peroxides, based on the weight of the oral care composition. In at least one embodiment, the oral care composition contains about 0 wt. % or 0 wt. % of complexed peroxides, based on the weight of the oral care composition.

The oral care compositions may preferably have a reduced amount polysaccharides and/or natural gums. For example, the oral care composition may preferably have about 4 wt. % or less, about 3 wt. % or less, about 2 wt. % or less, about 1 wt. % or less, about 0.5 wt. % or less, or about 0.1 wt. % or less, based on the weight of the oral care composition, of polysaccharides and/or natural gums. In at least one embodiment, the oral care composition contains about 0 wt. % or 0 wt. % of polysaccharides and/or natural gums, based on the weight of the oral care composition.

Additionally or alternatively, the oral care compositions may have a reduced amount thickening agents other than polyacrylic acid and/or a salt thereof, such as polysaccharides and/or natural gums. For example, the oral care composition may preferably have about 4 wt. % or less, about 3 wt. % or less, about 2 wt. % or less, about 1 wt. % or less, about 0.5 wt. % or less, or about 0.1 wt. % or less, based on the weight of the oral care composition, of polysaccharides and/or natural gums. In some embodiments, the oral care composition may preferably have a total amount of polysaccharides and natural gums of about 4 wt. % or less, about 3 wt. % or less, about 2 wt. % or less, about 1 wt. % or less, about 0.5 wt. % or less, or about 0.1 wt. % or less, based on the weight of the oral care composition.

Preferably, the oral care composition may achieve the stain resistance benefits while simultaneously have a desirable relative dental abrasivity. In certain embodiments, the oral care composition has a relative dental abrasivity of about 250 or less, about 200 or less, about 150 or less, about 125 or less, about 100 or less, or about 75 or less, as determined pursuant to standards DIN EN ISO 11609. Additionally or alternatively, the oral care composition may have about 10 or more, about 50 or more, or about 75 or more, as determined pursuant to standards DIN EN ISO 11609. For example, the oral care composition may have a relative dental abrasivity of about 10 to about 250, about 50 to about 200, or any other range formed from the disclosure herein, as determined pursuant to standards DIN EN ISO 11609.

The oral care compositions may be in the form of a dentifrice, a toothpaste, a gel, and/or the like. Suitable components, such as those listed below, may be included or excluded from the formulations for the oral care compositions disclosed herein depending on the specific combination of other ingredients and/or the form of the oral care compositions. Additionally or alternatively, the oral care compositions may in some embodiments have a single phase, which contains that components and/or ingredients of the oral care composition. In other embodiments, the oral care composition may include two or more phases, such as two, three, four, or five phases.

The oral care compositions include one or more whitening agent(s), preferably in an effective amount. The total amount of whitening agent(s) present in the oral care composition may be from about 0.1 to about 30 wt. %, based on the total weight of the oral care composition. For example, the whitening agent(s) may be present in the oral care composition in an amount from about 0.1 to about 10 wt. %, from about 0.1 to about 8 wt. %, from about 0.1 to about 6 wt. %, from about 0.1 to about 4 wt. %, from about 0.1 to about 3 wt. %, from about 0.1 to about 2 wt. %, from about 0.1 to about 1 wt. %; from about 1 to about 26 wt. %, about 1 to about 22 wt. %, about 1 to about 19 wt. %, about 1 to about 16 wt. %, about 1 to about 13 wt. %, about 1 to about 10 wt. %, about 1 to about 8 wt. %, about 1 to about 6 wt. %, about 1 to about 5 wt. %, about 1 to about 4 wt. %, about 1 to about 3 wt. %, about 1 to about 2 wt. %; from about 2 to about 30 wt. %, about 2 to about 26 wt. %, about 2 to about 22 wt. %, about 2 to about 19 wt. %, about 2 to about 16 wt. %, about 2 to about 13 wt. %, about 2 to about 10 wt. %, about 2 to about 8 wt. %, about 2 to about 6 wt. %, about 2 to about 5 wt. %, about 2 to about 4 wt. %; from about 3 to about 30 wt. %, about 3 to about 26 wt. %, about 3 to about 22 wt. %, about 3 to about 19 wt. %, about 3 to about 16 wt. %, about 3 to about 13 wt. %, about 3 to about 10 wt. %, about 3 to about 8 wt. %, about 3 to about 6 wt. %; from about 5 to about 30 wt. %, about 5 to about 26 wt. %, about 5 to about 22 wt. %, about 5 to about 19 wt. %, about 5 to about 16 wt. %, about 5 to about 13 wt. %, about 5 to about 10 wt. %, about 5 to about 8 wt. %; from about 8 to about 30 wt. %, about 8 to about 26 wt. %, about 8 to about 22 wt. %, about 8 to about 19 wt. %, about 8 to about 16 wt. %, about 8 to about 13 wt. %; from about 11 to about 30 wt. %, about 11 to about 26 wt. %, about 11 to about 22 wt. %, about 11 to about 19 wt. %, about 11 to about 16 wt. %; from about 14 to about 30 wt. %, about 14 to about 26 wt. %, about 14 to about 22 wt. %, about 14 to about 19 wt. %; from about 18 to about 30 wt. %, about 18 to about 26 wt. %, about 18 to about 22 wt. %, or any range or subrange thereof, based on the total weight of the oral are composition.

The whitening agent typically comprises from about 0.1 to about 10 wt. % of a peroxide. The total amount of peroxide in the oral care composition may be from about 0.1 to about 10 wt. %, from about 0.1 to about 8 wt. %, from about 0.1 to about 6 wt. %, from about 0.1 to about 4 wt. %, from about 0.1 to about 3 wt. %, from about 0.1 to about 2 wt. %, from about 0.1 to about 1 wt. %; from about 2 to about 10 wt. %, about 2 to about 8 wt. %, about 2 to about 7 wt. %, about 2 to about 6 wt. %, about 2 to about 5 wt. %, about 2 to about 4 wt. %; from about 3 to about 10 wt. %, about 3 to about 8 wt. %, about 3 to about 7 wt. %, about 3 to about 6 wt. %, about 3 to about 5 wt. %, about 3 to about 4 wt. %; from about 3.5 to about 10 wt. %, about 3.5 to about 8 wt. %, about 3.5 to about 7 wt. %, about 3.5 to about 6 wt. %, about 3.5 to about 5 wt. %, about 3.5 to about 4 wt. %; from about 4 to about 10 wt. %, about 4 to about 8 wt. %, about 4 to about 7 wt. %, about 4 to about 6 wt. %; from about 5 to about 10 wt. %, about 5 to about 8 wt. %, about 5 to about 7 wt. %; from about 6 to about 10 wt. %, about 6 to about 8 wt. %, or any range or subrange thereof, based on the total weight of the oral are composition.

The whitening agent(s) typically comprise a peroxide source, such as hydrogen peroxide, peroxides of alkali and alkaline earth metals, organic peroxy compounds, peroxy acids, salts thereof, or combinations of two or more thereof. The peroxide source may include peroxides of alkali and alkaline earth metals include lithium peroxide, potassium peroxide, sodium peroxide, magnesium peroxide, calcium peroxide, barium peroxide, and a combination of two or more thereof. Examples of organic peroxy compounds include carbamide peroxide (also known as urea hydrogen peroxide), glyceryl hydrogen peroxide, alkyl hydrogen peroxides, dialkyl peroxides, alkyl peroxy acids, peroxy esters, diacyl peroxides, benzoyl peroxide, monoperoxyphthalate, or a combination of two or more thereof. Peroxy acids and their salts include organic peroxy acids, such as alkyl peroxy acids, monoperoxyphthalate, and a combination thereof as well as inorganic peroxy acid salts, such as persulfate, dipersulfate, percarbonate, perphosphate, perborate and persilicate salts of alkali and/or alkaline earth metals, such as lithium, potassium, sodium, magnesium, calcium and barium, and mixtures thereof. In some embodiments, the peroxide is selected from hydrogen peroxide, lithium peroxide, potassium peroxide, sodium peroxide, magnesium peroxide, calcium peroxide, barium peroxide, alkyl hydrogen peroxides, dialkyl peroxides, alkyl peroxy acids, peroxy esters, diacyl peroxides, benzoyl peroxide, monoperoxyphthalate, linear PVP complexed with hydrogen peroxide, cross-linked PVP complexed with hydrogen peroxide, silica complexed with hydrogen peroxide, tetrasodium pyrophosphate, complexed with hydrogen peroxide, and a combination of two or more thereof. In various embodiments, the peroxide compound comprises hydrogen peroxide, urea peroxide, sodium percarbonate, or a combination of two or more thereof.

The one or more peroxide(s) may be a non-complexed peroxide. The non-complexed peroxide may be chosen from one or more of the peroxides described above. In some embodiments, the whitening agent consists of non-complexed peroxides. In at least one preferred embodiments, the non-complexed peroxide comprises hydrogen peroxide. In another embodiment, the whitening agent consists of hydrogen peroxide.

In some embodiments, however, the whitening agent may comprise a peroxide complex. The peroxide complex may comprise a peroxide component and a porous cross-linked polymer. As referred to herein, a “peroxide component” is any oxidizing compound comprising a bivalent oxygen-oxygen group. Peroxide components include peroxides and hydroperoxides disclosed above, such as hydrogen peroxide, peroxides of alkali and alkaline earth metals, organic peroxy compounds, peroxy acids, salts thereof, and mixtures thereof. In various embodiments, the peroxide component comprises hydrogen peroxide, urea peroxide, sodium percarbonate and mixtures thereof. In one embodiment, the peroxide component comprises hydrogen peroxide. In one embodiment, the peroxide component consists essentially of hydrogen peroxide.

The peroxide complex may also be selected from N-vinyl heterocyclic polymers. Preferably, the N-vinyl heterocyclic polymer is crosslinked and adsorbs, absorbs, complexes, or otherwise retains the peroxide component. The chemical and physical characteristics of the particulate hinder the release of the peroxide compound from the polymer particulates, and in some embodiments provides controlled release of the peroxide compound. The peroxide complex preferably comprises a peroxide component at a level of from about 0.1 to about 25 wt. %, from about 1 to about 25 wt. %, from about 5 to about 18 wt. %, based on the weight of the peroxide complex. In some preferred embodiments, the oral care composition comprises a complex of peroxide adsorbed to cross-linked polyvinylpyrrollidone.

In some embodiments, a non-peroxide whitening agent may be included in the oral care composition. Whitening agents among those useful herein include non-peroxy compounds, such as chlorine dioxide, chlorites and hypochlorites. Chlorites and hypochlorites include those of alkali and alkaline earth metals such as lithium, potassium, sodium, magnesium, calcium and barium. Non-peroxide whitening agents also include colorants, such as titanium dioxide and hydroxyapatite, pigments or dyes.

The oral care compositions typically comprise one or more cross-linked polyacrylic acid and/or a salt thereof. The one or more cross-linked acrylic acid and/or a salt thereof may be present in an amount from about 0.2 to about 5 wt. %, based on the total weight of the oral care composition. In some preferred instances, the one or more cross-linked acrylic acid and/or a salt may be present in an amount from about 0.2 to about 4 wt. %, about 0.2 to about 3 wt. %, about 0.2 to about 2.5 wt. %, about 0.2 to about 2 wt. %, about 0.2 to about 1.5 wt. %, about 0.2 to about 1.25 wt. %, about 0.2 to about 1 wt. %, about 0.2 to about 0.75 wt. %, about 0.2 to about 0.5 wt. %; from about 0.35 to about 5 wt. %, about 0.35 to about 4 wt. %, about 0.35 to about 3 wt. %, about 0.35 to about 2.5 wt. %, about 0.35 to about 2 wt. %, about 0.35 to about 1.5 wt. %, about 0.35 to about 1.25 wt. %, about 0.35 to about 1 wt. %, about 0.35 to about 0.75 wt. %, about 0.35 to about 0.5 wt. %; from about 0.5 to about 5 wt. %, about 0.5 to about 4 wt. %, about 0.5 to about 3 wt. %, about 0.5 to about 2.5 wt. %, about 0.5 to about 2 wt. %, about 0.5 to about 1.5 wt. %, about 0.5 to about 1.25 wt. %, about 0.5 to about 1 wt. %, about 0.5 to about 0.75 wt. %; from about 0.75 to about 5 wt. %, about 0.75 to about 4 wt. %, about 0.75 to about 3 wt. %, about 0.75 to about 2.5 wt. %, about 0.75 to about 2 wt. %, about 0.75 to about 1.5 wt. %, about 0.75 to about 1.25 wt. %, about 0.75 to about 1 wt. %; from about 1 to about 5 wt. %, about 1 to about 4 wt. %, about 1 to about 3 wt. %, about 1 to about 2.5 wt. %, about 1 to about 2 wt. %, about 1 to about 1.5 wt. %, about 1 to about 1.25 wt. %; from about 1.5 to about 5 wt. %, about 1.5 to about 4 wt. %, about 1.5 to about 3 wt. %, about 1.5 to about 2.5 wt. %, about 1.5 to about 2 wt. %; from about 2 to about 5 wt. %, about 2 to about 4 wt. %, about 2 to about 3 wt. %, about 2 to about 2.5 wt. %; from about 2.5 to about 5 wt. %, about 2.5 to about 4 wt. %, about 2.5 to about 3 wt. %, about 3 to about 4 wt. %; from about 3 to about 5 wt. %, about 3 to about 4 wt. %, about 3 to about 3.5 wt. %, or any range or subrange thereof, based on the total weight of the oral care composition.

The cross-linked polyacrylic acid and/or a salt thereof may be selected from branched polyacrylic acids and/or salts thereof. In some instances, the polyacrylic acid and/or a salt thereof consists of branched polyacrylic acids and/or salts thereof. In further instances, however, the oral care composition includes at least one linear polyacrylic acid and/or a salt thereof.

The cross-linked polyacrylic acid and/or a salt thereof may be a carbomer. The carbomer may comprise or be selected from the group consisting of Carbopols. For instance, the personal care composition may include, or in some cases exclude, a polyacrylic acid and/or a salt thereof selected from Carbopol 2984, Carbopol 934, Carbopol Ultrez 10, Carbopol 940, Carbopol 941, Carbopol 956 Carbopol 981, Carbopol 2020, and a combination of two or more thereof. In some cases, the polyacrylic acid and/or a salt thereof comprises one or more of or is selected from the group consisting from Carbopol 2984, Carbopol 910, Carbopol 934P, Carbopol Ultrez 10, Carbopol 940, Carbopol 941, Carbopol 956, Carbopol 971P, Carbopol 974P, Carbopol 981, Carbopol 2020, and/or a combination of two or more thereof. Additionally or alternatively, the cross-linked polyacrylic acid and/or a salt thereof may be selected from carbomers. In at least one embodiment, the carbomer is CARBOIER 974P. In at least one embodiment, the carbomer comprises or consists of carbopol ETD NF 2020. In some instances, the cross-linked polyacrylic acid and/or the salt thereof consists of one or more carbomer(s).

The oral care compositions may include water, typically in an amount up to about 10 wt. % based on the total weight of the oral care composition. For instance, in some embodiments, the oral care compositions include up to about 8 wt. %, up to about 7 wt. %, up to about 6 wt. %, up to about 5 wt. %, up to about 4 wt. %, up to about 3 wt. %, up to about 2 wt. %, or up to about 1 wt. % of water, based on the total weight of the oral care composition. In some embodiments, the oral care composition comprises water in an amount from about 0.5 to about 8 wt. %, about 0.5 to about 7 wt. %, about 0.5 to about 6 wt. %, about 0.5 to about 5 wt. %, about 0.5 to about 4 wt. %, about 0.5 to about 3 wt. %, about 0.5 to about 2 wt. %; from about 1 to about 8 wt. %, about 1 to about 7 wt. %, about 1 to about 6 wt. %, about 1 to about 5 wt. %, about 1 to about 4 wt. %, about 1 to about 3 wt. %, about 1 to about 2 wt. %; from about 2 to about 10 wt. %, about 2 to about 8 wt. %, about 2 to about 7 wt. %, about 2 to about 6 wt. %, about 2 to about 5 wt. %, about 2 to about 4 wt. %, about 2 to about 3 wt. %; from about 3 to about 10 wt. %, about 3 to about 8 wt. %, about 3 to about 7 wt. %, about 3 to about 6 wt. %, about 3 to about 5 wt. %, about 3 to about 4 wt. %; from about 4 to about 10 wt. %, about 4 to about 8 wt. %, about 4 to about 7 wt. %, about 4 to about 6 wt. %; from about 5 to about 10 wt. %, about 5 to about 8 wt. %, about 5 to about 7 wt. %, about 6 to about 10 wt. %, about 6 to about 8 wt. %, based on the total weight of the oral care composition.

The oral care compositions typically have one or more polyol. The amount of polyol(s) in the oral care compositions may be from about 30 to about 70 wt. %, based on the total weight of the oral care composition. For example, the total amount of polyol(s) in the oral care compositions may from about 30 to about 70 wt. %, about 30 to about 66 wt. %, about 30 to about 62 wt. %, about 30 to about 58 wt. %, about 30 to about 54 wt. %, about 30 to about 50 wt. %, about 30 to about 46 wt. %, about 30 to about 42 wt. %; from about 35 to about 70 wt. %, about 35 to about 66 wt. %, about 35 to about 62 wt. %, about 35 to about 58 wt. %, about 35 to about 54 wt. %, about 35 to about 50 wt. %, about 35 to about 46 wt. %; from about 40 to about 70 wt. %, about 40 to about 66 wt. %, about 40 to about 62 wt. %, about 40 to about 58 wt. %, about 40 to about 54 wt. %, about 40 to about 50 wt. %; from about 44 to about 70 wt. %, about 44 to about 66 wt. %, about 44 to about 62 wt. %, about 44 to about 58 wt. %, about 44 to about 54 wt. %; from about 48 to about 70 wt. %, about 48 to about 66 wt. %, about 48 to about 62 wt. %, about 48 to about 58 wt. %; from about 52 to about 70 wt. %, about 52 to about 66 wt. %, about 52 to about 62 wt. %; from about 56 to about 70 wt. %, about 56 to about 66 wt. %, from about 58 to about 70 wt. %, about 58 to about 66 wt. %; from about 60 to about 70 wt. %, about 60 to about 66 wt. %, based on the total weight of the oral care composition.

The polyol(s) may be chosen from glycols or compounds with numerous hydroxyl groups. The one or more polyols may be liquid at ambient temperature (25° C.). In some preferred embodiments, the polyol(s) comprises glycerin, glycol, inositol, maltitol, mannitol, sorbitol, xylitol, propylene glycol, polypropylene glycol (PPG), polyethylene glycol (PEG), a block copolymer of PPG and PEG, a saccharide (e.g., fructose, glucose, sucrose and mixtures of saccharides, such as honey), or a combination of two or more thereof. For instance, the oral care composition may comprise maltitol, mannitol, sorbitol, xylitol, a polypropylene glycol (PPG), a polyethylene glycol (PEG), a block copolymer of PPG and PEG, or a combination or two or more thereof. The polyol may be a humectant including, e.g., glycol, sorbitol, glycerin, inositol, maltitol, mannitol, xylitol, propylene glycol, polypropylene glycol (PPG), polyethylene glycol (PEG), a saccharide, polyoxypropylene-polyoxyethylene block copolymer, and a combination of two or more thereof.

In some cases, the oral care composition includes one or more polyol selected from the group consisting of C₂-C₃₂ polyols. The one or more polyols may have from 2 to 32 carbon atoms, from 3 to 16 carbon atoms, or from 3 to 12 carbon atoms. For example, the oral care composition may comprise ethylene glycol, propylene glycol, butylene glycol, hexylene glycol, glycerin, diglycerin, diethylene glycol, and dipropylene glycol, or a combination of two or more thereof. Additional, non-limiting examples of polyols that may, optionally, be included in the oral care include and/or may be chosen from alkanediols such as glycerin, 1,2,6-hexanetriol, trimethylolpropane, ethylene glycol, propylene glycol, butylene glycol, pentylene glycol, hexylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, caprylyl glycol, 1,2-hexanediol, 1,2-pentanediol, and 4-methyl-1,2-pentanediol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, dipropylene glycol mono-iso-propyl ether, sorbitol, sorbitan, triacetin, and a mixture of two or more thereof.

Additionally or alternatively, the oral care composition may include polyol(s) having a molecular weight of from about 100 to 5000 g/mol. For instance, the polyol may comprise a polyethylene glycol, a polypropylene glycol, a block polymer of polyethylene glycol and polypropylene glycol, or a combination of two or more thereof. In some embodiments, the polyol comprises a polypropylene glycol, a polypropylene glycol, and/or a block polymer of polyethylene glycol and polypropylene glycol having a molecular weight of about 100 to about 900, about 200 to about 800, about 400, about 1500 to about 2500, about 2000 to about 4500 or any range or subrange thereof. In some embodiments, the polyol is a polyethylene glycol, such as polyethylene glycol 600. In some embodiments, the oral care composition comprises one or more polyethylene glycols, e.g., polyethylene glycols in a molecular weight range from 200 to 800. For example, the compositions may comprise one or more of polyethylene glycol 200, polyethylene glycol 300, polyethylene glycol 400, polyethylene glycol, 600 or polyethylene glycol 800.

The oral care composition may comprise a plurality of polyols. In some embodiments, at least one of the plurality of polyols is selected from glycerin, sorbitol, ethylene glycol, propylene glycol, butylene glycol, pentylene glycol, hexylene glycol, diethylene glycol, triethylene glycol, tetraethylene glycol, pentaethylene glycol, dipropylene glycol, a polyethylene glycol, a polypropylene glycol, a block polymer of polyethylene glycol and polypropylene glycol, and a combination of two or more thereof. In some instances, at least one of the polyols is selected from propylene glycol and polyethylene glycol-polypropylene glycol copolymer. In at least one embodiment, the oral care composition comprises propylene glycol and polyethylene glycol-polypropylene glycol copolymer.

The amount of abrasive(s) present in the oral are composition may be from about 1 to about 35 wt. %, based on the total weight of the oral care composition. In some instances, the total amount of abrasive(s) present in the oral care composition is from about 1 to about 35 wt. %, about 1 to about 35 wt. %, about 1 to about 31 wt. %, about 1 to about 27 wt. %, about 1 to about 23 wt. %, about 1 to about 19 wt. %, about 1 to about 17 wt. %, about 1 to about 15 wt. %, about 1 to about 13 wt. %; from about 4 to about 35 wt. %, about 4 to about 35 wt. %, about 4 to about 31 wt. %, about 4 to about 27 wt. %, about 4 to about 23 wt. %, about 4 to about 19 wt. %, about 4 to about 17 wt. %, about 4 to about 15 wt. %, about 4 to about 13 wt. %; from about 7 to about 35 wt. %, about 7 to about 35 wt. %, about 7 to about 31 wt. %, about 7 to about 27 wt. %, about 7 to about 23 wt. %, about 7 to about 19 wt. %, about 7 to about 17 wt. %, about 7 to about 15 wt. %, about 7 to about 13 wt. %; from about 10 to about 35 wt. %, about 10 to about 35 wt. %, about 10 to about 31 wt. %, about 10 to about 27 wt. %, about 10 to about 23 wt. %, about 10 to about 19 wt. %, about 10 to about 17 wt. %, about 10 to about 15 wt. %; from about 13 to about 35 wt. %, about 13 to about 35 wt. %, about 13 to about 31 wt. %, about 13 to about 27 wt. %, about 13 to about 23 wt. %, about 13 to about 19 wt. %, about 13 to about 17 wt. %, about 13 to about 15 wt. %; from about 15 to about 35 wt. %, about 15 to about 35 wt. %, about 15 to about 31 wt. %, about 15 to about 27 wt. %, about 15 to about 23 wt. %, about 15 to about 19 wt. %; from about 17 to about 35 wt. %, about 17 to about 35 wt. %, about 17 to about 31 wt. %, about 17 to about 27 wt. %, about 17 to about 23 wt. %; from about 19 to about 35 wt. %, about 19 to about 35 wt. %, about 19 to about 31 wt. %, about 19 to about 27 wt. %, about 19 to about 23 wt. %, or any range or subrange thereof, based on the total weight of the oral care composition.

The abrasive may comprise an insoluble phosphate salts, such as those chosen from calcium pyrophosphate, dicalcium orthophosphate dihydrate, tricalcium phosphate, and calcium polymetaphosphate, and a combination of two or more thereof. In some embodiments, the abrasive is a calcium based abrasive.

In some preferred embodiments, the oral care composition includes a calcium-based abrasive. Examples of calcium based abrasives include calcium pyrophosphate, dicalcium orthophosphate dihydrate, tricalcium phosphate, calcium polymetaphosphate, calcium carbonate, calcium bicarbonate, or a combination thereof. The calcium-based abrasive may be selected from calcium pyrophosphate, dicalcium orthophosphate dihydrate, tricalcium phosphate, calcium polymetaphosphate, and a combination of two or more thereof. In at least one embodiment, the abrasive consists of one or more calcium-based abrasive(s).

The abrasive of the oral care composition may be comprised primarily of calcium pyrophosphate and, in at least one embodiment, calcium pyrophosphate is the sole abrasive in the oral care composition. For example, the calcium pyrophosphate may comprise about 50 wt. % or more, about 60 wt. % or more, about 70 wt. % or more, about 80 wt. % or more, about 95 wt. % or more, about 98 wt. % or more, or about 99 wt. % or more of the total amount of the abrasive in the oral care composition.

Additional abrasives worth mentioning, which may be included or excluded from the oral care composition include: silica, silicate, silicon, alumina (including calcined aluminum oxide), aluminosilicates, such as bentonite, zeolite, kaolin, and mica, siliceous or diatomaceous earth, pumice, calcium carbonate, cuttlebone, insoluble phosphates, composite resins, such as melamine resin, phenolic resin, and urea-formaldehyde resin, polycarbonate, silicon carbide, boron carbide, microcrystalline wax, microcrystalline cellulose, including combinations of colloidal microcrystalline cellulose and carboxymethylcellulose, and combinations and derivatives thereof.

As used herein, “mica” refers to any of a group of hydrous aluminum silicate minerals with plate morphology and/or perfect basal (micaceous) cleavage. Mica can be, for example, sheet mica, scrap mica or flake mica, as exemplified by muscovite, biotite or phlogopite type micas. The abrasive may be selected from insoluble phosphates, such as orthophosphates, polymetaphosphates, pyrophosphates, and a combination thereof. Synthetic silicas include both silica gels and precipitated silicas that are prepared by the neutralization of aqueous silicate solutions with a strong mineral acid. Abrasives comprising silica may be useful in certain embodiments of the oral care composition. In certain embodiments, the oral compositions may comprise a particularly efficacious combination of silica abrasive particle species. For example, the abrasive(s) be selected from high cleaning silica, tetrasodium pyrophosphate (TSPP), sodium tripolyphosphate (STPP), and a combination of two or more thereof.

The oral care compositions may include an abrasive system comprising two or more abrasives. For example, an abrasive system comprise 2 to 7, 2 to 6, 2 to 5, 2 to 5, or 2 to 4; 3 to 7, 3 to 6, 2 to 5, or 3 to 5 abrasives. The abrasives may comprise one or more cleaning abrasive and/or one or more polishing abrasives. As appreciated by one of skill in the art, a single abrasive species typically performs at least some cleaning and polishing simultaneously. However, particles are generally categorized in the art by the predominant effect they have on a target oral surface. Typically, “polishing abrasives” are considered to be relatively small particles having high hardness, where abrasives with relatively large particle sizes and low hardness are considered to be “cleaning abrasives.” In certain embodiments, the oral care composition comprises two or more abrasives comprising silica. In some embodiments, the first abrasive is selected to be a harder and smaller abrasive, e.g., a higher cleaning and/or polishing abrasive, and the second abrasive is a typical cleaning abrasive. In some embodiments, the oral care composition includes at least one polishing abrasive and/or at least one cleaning abrasive. Further examples of abrasives are disclosed in U.S. Patent Publication No. 2007/140986, which is incorporated herein in its entirety by reference for all purposes.

The oral care compositions may include an anti-calculus agent. The amount of anti-calculus agent(s) present in the oral are composition may be from about 0.5 to about 10 wt. %, based on the total weight of the oral care composition. In some instances, the anti-calculus agent(s) is present in the oral care composition in an amount from about 0.5 to about 10 wt. %, about 0.5 to about 8 wt. %, about 0.5 to about 6 wt. %, about 0.5 to about 5 wt. %, about 0.5 to about 4 wt. %, about 0.5 to about 3 wt. %, about 0.5 to about 2 wt. %; from about 1 to about 10 wt. %, about 1 to about 8 wt. %, about 1 to about 6 wt. %, about 1 to about 5 wt. %, about 1 to about 4 wt. %, about 1 to about 3 wt. %, about 1 to about 2 wt. %; from about 2 to about 10 wt. %, about 2 to about 8 wt. %, about 2 to about 6 wt. %, about 2 to about 5 wt. %, about 2 to about 4 wt. %, about 2 to about 3 wt. %; from about 3 to about 10 wt. %, about 3 to about 8 wt. %, about 3 to about 6 wt. %, about 3 to about 5 wt. %, about 3 to about 4 wt. %; from about 5 to about 10 wt. %, about 5 to about 8 wt. %, about 5 to about 6 wt. %; from about 7 to about 10 wt. %, about 7 to about 8 wt. %, about 8 to about 10 wt. %, or any range or subrange thereof, based on the total weight of the oral care composition.

The anti-calculus agent(s) may be chosen from phosphates and polyphosphates (e.g., pyrophosphates), polyaminopropanesulfonic acid (AMPS), hexametaphosphate salts, zinc citrate trihydrate, polypeptides, polyolefin sulfonates, polyolefin phosphates, diphosphonates, and combinations of two or more thereof. Examples of anti-calculus agents include tetrasodium pyrophosphate, ammonium tripolyphosphate, hexametaphosphate salt, polycarboxylate polymers, polyvinyl methyl ether/maleic anhydride (PVME/MA) copolymers, or combinations of two or more thereof. In some embodiments, the oral care composition includes one or more alkali metal polyphosphate, e.g., as tetrasodium pyrophosphate.

The oral care compositions may include a fluoride ion source. The fluoride ion source may be present in an effective amount. In some cases, the fluoride ion source is present in the oral care composition in an amount of about 0.1 to about 7 wt. %, based on the total weight of the oral care composition. For example, the fluoride ion source may be present in an amount of about 0.1 to about 7 wt. %, about 0.1 to about 5 wt. %, about 0.1 to about 3 wt. %, about 0.1 to about 2 wt. %, about 0.1 to about 1 wt. %; from about 0.4 to about 7 wt. %, about 0.4 to about 5 wt. %, about 0.4 to about 3 wt. %, about 0.4 to about 2 wt. %, about 0.4 to about 1 wt. %; from about 0.8 to about 7 wt. %, about 0.8 to about 5 wt. %, about 0.8 to about 3 wt. %, about 0.8 to about 2 wt. %, about 0.8 to about 1.5 wt. %; from about 1.4 to about 7 wt. %, about 1.4 to about 5 wt. %, about 1.4 to about 3 wt. %; from about 2 to about 7 wt. %, about 2 to about 5 wt. %, about 2 to about 3 wt. %; from about 3 to about 7 wt. %, about 3 to about 5 wt. %, about 3 to about 4 wt. %; from about 4 to about 7 wt. %, about 4 to about 6 wt. %, about 4 to about 5 wt. %; from about 5 to about 7 wt. %, about 5 to about 6 wt. %, or any range or subrange thereof, based on the total weight of the oral care composition.

The fluoride ion source may be selected from soluble fluoride ion salts. For example, the fluoride ion source may comprise sodium fluoride, potassium fluoride, calcium fluoride, zinc fluoride, zinc ammonium fluoride, lithium fluoride, ammonium fluoride, stannous fluoride, stannous fluorozirconate, sodium monofluorophosphate, potassium monofluorophosphate, laurylamine hydrofluoride, diethylaminoethyloctoylamide hydrofluoride, didecyldimethylammonium fluoride, cetylpyridinium fluoride, dilaurylmorpholinium fluoride, sarcosine stannous fluoride, glycine potassium fluoride, glycine hydrofluoride, amine fluorides or a combination of two or more thereof. In some embodiments, the fluoride ion source comprises sodium fluoride, sodium monofluorophosphate, or a combination thereof. Additional examples of fluoride ion sources are disclosed in U.S. Pat. Nos. 3,535,421, 4,885,155, and 3,678,154, the disclosures of which are hereby incorporated by reference in their entirety for all purposes.

The oral care compositions may include a desensitizing agent. The amount of desensitizing agent may be from about 0.5 to about 9 wt. %, based on the total weight of the oral care composition. For example, the amount of desensitizing agent present in the oral care composition may be from about 0.5 to about 8 wt. %, about 0.5 to about 6 wt. %, about 0.5 to about 5 wt. %, about 0.5 to about 4 wt. %, about 0.5 to about 3 wt. %, about 0.5 to about 2 wt. %; from about 1 to about 9 wt. %, about 1 to about 8 wt. %, about 1 to about 6 wt. %, about 1 to about 5 wt. %, about 1 to about 4 wt. %, about 1 to about 3 wt. %, about 1 to about 2 wt. %; from about 2 to about 9 wt. %, about 2 to about 8 wt. %, about 2 to about 6 wt. %, about 2 to about 5 wt. %, about 2 to about 4 wt. %, about 2 to about 3 wt. %; from about 3 to about 9 wt. %, about 3 to about 8 wt. %, about 3 to about 6 wt. %, about 3 to about 5 wt. %, about 3 to about 4 wt. %; from about 4 to about 9 wt. %, about 4 to about 8 wt. %, about 4 to about 6 wt. %, about 4 to about 5 wt. %; from about 5 to about 9 wt. %, about 5 to about 8 wt. %, about 5 to about 6 wt. %; from about 6 to about 9 wt. %, about 6 to about 8 wt. %; from about 7 to about 9 wt. %, about 7 to about 8 wt. %, about 8 to about 10 wt. %, or any range or subrange therefrom, based on the total weight of the oral care composition.

The desensitizing agent(s) may comprise, e.g., benzocaine, potassium nitrate; potassium fluoride; strontium chloride; potassium chloride; potassium citrate; iron oxalate; sodium nitrate; lithium nitrate; magnesium nitrate; calcium nitrate; calcium hydroxide; dibasic calcium phosphate; strontium acetate; sodium monofluorophosphate; bisabolol; a local or systemic analgesic agent such as NSAIDS, aspirin, acetaminophen and/or codeine; or combinations of two or more thereof. For example, the desensitizing agent(s) may be chosen from potassium nitrate, potassium citrate, potassium chloride, potassium bicarbonate, potassium oxalate, and a combination of two or more thereof. In some embodiments, the desensitizing agent is potassium nitrate.

The oral care composition may include one or more thickening agent(s) other than the polyacrylic acid and/or a salt thereof. The amount of thickening agent(s) other than polyacrylic acid and/or a salt thereof may be from about 0.1 to about 8 wt. %, based on the total weight of the oral care composition. For example, the amount of thickening agent(s) other than polyacrylic acid and/or a salt thereof in the oral care composition may be from about 0.1 to about 6 wt. %, about 0.1 to about 4 wt. %, about 0.1 to about 3 wt. %, about 0.1 to about 2 wt. %; from about 0.3 to about 5 wt. %, about 0.3 to about 4 wt. %, about 0.3 to about 3 wt. %, about 0.3 to about 2 wt. %; from about 0.6 to about 5 wt. %, about 0.6 to about 4 wt. %, about 0.6 to about 3 wt. %, about 0.6 to about 2 wt. %; from about 0.9 to about 5 wt. %, about 0.9 to about 4 wt. %, about 0.9 to about 3 wt. %, about 0.9 to about 2 wt. %; from about 1.2 to about 5 wt. %, about 1.2 to about 4 wt. %, about 1.2 to about 3 wt. %, about 1.2 to about 2 wt. %; from about 1.5 to about 5 wt. %, about 1.5 to about 4 wt. %, about 1.5 to about 3 wt. %; from about 2 to about 8 wt. %, about 2 to about 6 wt. %, about 2 to about 5 wt. %, about 2 to about 4 wt. %, about 2 to about 3 wt. %; from about 3 to about 8 wt. %, about 3 to about 6 wt. %, about 3 to about 5 wt. %; from about 4 to about 8 wt. %, about 4 to about 6 wt. %, about 4 to about 5 wt. %; from about 5 to about 8 wt. %, about 5 to about 6 wt. %, or any range or subrange thereof, based on the total weight of the oral care composition.

As mentioned above, in certain embodiments, the oral care composition may have a reduced amount of thickening agents other than polyacrylic acid and/or a salt thereof. For instance, the oral care composition may comprise thickening agents other than polyacrylic acid and/or a salt thereof in an amount from about 0.1 to about 2.5 wt. % about 0.3 to about 2.5 wt. %, about 0.6 to about 2 wt. %, about 1 to about 2.5 wt. %; from about 0.1 to about 2 wt. % about 0.3 to about 2 wt. %, about 0.6 to about 2 wt. %, about 1 to about 2 wt. %; from about 0.1 to about 1.5 wt. % about 0.3 to about 1.5 wt. %, about 0.6 to about 1.5 wt. %, or any range or subrange thereof from about 0.1 to about 1 wt. % about 0.3 to about 1 wt. %, about 0.6 to about 1 wt. %, or any range or subrange thereof, based on the total weight of the oral care composition.

Thickening agents may be referred to as “thickeners” or “viscosity modifying agents.” Thickening agents are typically included to increase the viscosity of the oral care compositions. Nonetheless, in some instances, certain thickening agents provide additional, surprising benefits to the oral care compositions.

The one or more thickening agent(s) other than polyacrylic acid and/or a salt thereof may be selected from a polysaccharide, a silica thickener, an acrylate, a polymer other than polyacrylic acid and/or a salt thereof, and a combination of two or more thereof. Examples of polysaccharides include cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, carrageenan, or a combination of two more thereof. Natural gums such as karaya, gum arabic, and gum tragacanth can also be incorporated. Colloidal magnesium aluminum silicate can also be used as a component of the thickening composition to further improve the composition's texture. Silica thickeners, which form polymeric structures or gels in aqueous media, may be present. Silica thickeners are typically physically and functionally distinct from the particulate silica abrasives as the silica thickeners are very finely divided and provide little or no abrasive action.

In some embodiments, the one or more thickening agent(s) comprises a polymer selected from polysaccharides, acrylates, polyvinyl pyrrolidone, polyitaconates, an acrylamides, and combinations thereof. Polyvinyl pyrrolidone generally refers to a polymer containing vinylpyrrolidone (e.g., N-vinylpyrrolidone, N-vinyl-2-pyrrolidione, and N-vinyl-2-pyrrolidinone) as a monomeric unit. The monomeric unit may include a polar imide group, four non-polar methylene groups, and a non-polar methane group. The polyvinyl pyrrolidone may have an average molecular weight in the range 5,000 to 100,000, preferably in the range 5,000 to 50,000. Polyvinyl pyrrolidones that have average molecular weights of 10,000, 30,000 and 40,000 may be commercially available from Sigma Chemical Co., GAF Corporation and Sigma Chemical Co. In some embodiments, the polymer comprises crosslinked polyvinyl pyrrolidone (crosslinked PVP). In at least one embodiment, the polymer consists of polyvinyl pyrrolidone.

Examples of acrylates that may be included in the oral care composition include, e.g., isobutyl acrylate, tert-butyl acrylate, 2-ethylhexyl acrylate, lauryl acrylate, lauryl/tridecyl acrylate, cetyl acrylate, stearyl acrylate, cyclohexyl acrylate, benzyl acrylate, isobornyl acrylate, 2-methoxyethyl acrylate, 2-ethoxyethyl acrylate, 2-ethoxyethoxyethyl acrylate, 2-phenoxyethyl acrylate, tetrahydrofurfuryl acrylate, 2-hydroxyethyl acrylate, 2-hydroxypropyl acrylate, 4-hydroxybutyl acrylate, dimethylaminoethyl acrylate, 1,4-butanediol acrylate, or a combination of two or more thereof.

The acrylate may be chosen from diacrylates. In some embodiments, the oral care composition includes a diacrylate chosen from 1,4-butanediol, 1,6-hexanediol, tetraethylene glycol, tripropylene glycol, ethoxylated bisphenol-A, and a combination of two or more thereof. Triacrylate monomers include trimethylol propane, ethoxylated, glyceryl propoxy, and pentaerythritol.

Acrylates further include methacrylates, such as methyl methacrylate, ethyl methacrylate, n-butyl methacrylate, isobutyl methacrylate, tert-butyl methacrylate, 2-ethylhexyl methacrylate, lauryl methacrylate, alkyl methacrylate, tridecyl methacrylate, stearyl methacrylate, cyclohexyl methacrylate, benzyl methacrylate, isobornyl methacrylate, 2-hydroxyethyl methacrylate, 2-hydroxypropyl methacrylate, dimethylaminoethyl methacrylate, diethylaminoethyl methacrylate, glycidyl methacrylate, tetrahydrofurfuryl methacrylate, allyl methacrylate, ethylene glycol methacrylate, triethylene glycol methacrylate, tetraethylene glycol methacrylate, 1,3-butyleneglycol methacrylate, 1,6-hexanediol methacrylate, trimethylopropane methacrylate, ethoxyethyl methacrylate, trifluoroethyl methacrylate, or a combination of two or more thereof.

Examples of acrylamides include, but are not limited to, acrylamide, methacrylamide and di(C₁-C₃₀) alkyl-acrylamides and -methacrylamides such as those of methyl, ethyl, propyl, butyl, pentyl, hexyl and the like. N-substituted acrylamides that may be suitable include N-ethylacrylamide, N-tert-butylacrylamide, N-tert-octylacrylamide, N-octylacrylamide, N-decylacrylamide, N-dodecylacrylamide and the corresponding N-substituted methacrylamides. Other N-substituted acrylamides include N-hydroxymethyl acrylamide, N-isopropylacrylamide, N-methylacrylamide, N,N′-methylenebisacrylamide, N-isobutoxymethylacrylamide, N,N-dimethylacrylamide, and 2-acrylamido-2-methylpropanesulfonic acid. In some embodiments, one or more of the above listed thickening agents other than polyacrylic acid and/or a salt thereof are excluded from the oral care composition.

The oral care compositions may include one or more surfactant(s) in various amounts. The oral care composition may comprise one or more anionic surfactant, one or more amphoteric surfactant, one or more cationic surfactant, one or more nonionic surfactant, or a combination of two or more thereof. For example, the oral care composition may include one or more anionic surfactant(s) in an amount of about 0.5 to about 10 wt. %, based on the total weight of the oral care composition. In some instances, the anionic surfactant(s) is present in the oral care composition in an amount from about 0.5 to about 10 wt. %, about 0.5 to about 8 wt. %, about 0.5 to about 6 wt. %, about 0.5 to about 5 wt. %, about 0.5 to about 4 wt. %, about 0.5 to about 3 wt. %, about 0.5 to about 2 wt. %; from about 1 to about 10 wt. %, about 1 to about 8 wt. %, about 1 to about 6 wt. %, about 1 to about 5 wt. %, about 1 to about 4 wt. %, about 1 to about 3 wt. %, about 1 to about 2 wt. %; from about 2 to about 10 wt. %, about 2 to about 8 wt. %, about 2 to about 6 wt. %, about 2 to about 5 wt. %, about 2 to about 4 wt. %, about 2 to about 3 wt. %; from about 3 to about 10 wt. %, about 3 to about 8 wt. %, about 3 to about 6 wt. %, about 3 to about 5 wt. %, about 3 to about 4 wt. %; from about 5 to about 10 wt. %, about 5 to about 8 wt. %, about 5 to about 6 wt. %; from about 7 to about 10 wt. %, about 7 to about 8 wt. %, about 8 to about 10 wt. %, or any range or subrange thereof, based on the total weight of the oral care composition.

The anionic surfactant(s) may be selected from sulfate based anionic surfactants and non-sulfate based anionic surfactants. For example, the oral care composition may include an anionic surfactant chosen from ammonium lauryl sulfate, ammonium lauryl ether sulfate, sodium dodecyl sulfate, sodium coco-sulfate, ammonium coco-sulfate, and a combination of two or more thereof.

In some preferred embodiments, the oral care composition comprises sodium lauryl sulfate, sodium lauryl ether sulfate, or a combination thereof. In further embodiments, one or more anionic surfactant is selected from sodium lauryl sulfate, sodium lauryl ether sulfate, sodium lauryl sarcosinate, sodium cocoyl methyl taurate, sodium monoglyceride sulfate, sodium cetaryl sulfate, potassium cocoyl glycinate, sodium lauryl phosphate, sodium lauryl lactylate, sodium lauryl sulfoacetate, sodium lauryl glutamate, sodium lauryl isethionate, sodium laureth carboxylate, sodium dodecyl benzenesulfonate, and a combination of two or more thereof.

The one or more anionic surfactant(s) may be selected from non-sulfate based anionic surfactants, such as sulfonated monoglycerides of fatty acids, acyl isethionates, acyl sarcosinates, acyl taurate, and a combination of two or more thereof. Non-limiting examples of acyl isethionates include sodium isethionate, sodium cocoyl isethionate, sodium lauroyl methyl isethionate, and sodium cocoyl methyl isethionate. Sulfonated monoglycerides of fatty acids include sodium coconut monoglyceride sulfonates and the like. Examples of acyl sarcosinates include potassium lauroyl sarcosinate, potassium cocoyl sarcosinate, sodium cocoyl sarcosinate, sodium lauroyl sarcosinate, sodium myristoyl sarcosinate, sodium oleoyl sarcosinate, sodium palmitoyl sarcosinate, ammonium lauroyl sarcosinate, and combinations of two or more thereof. Examples of acyl taurate surfactants include sodium cocoyl taurate, potassium cocoyl taurate, potassium methyl cocoyl taurate, sodium caproyl methyl taurate, sodium cocoyl taurate, sodium lauroyl taurate, sodium methyl cocoyl taurate (SMCT), sodium methyl lauroyl taurate, sodium methyl myristoyl taurate, sodium methyl oleoyl taurate, sodium methyl palmitoyl taurate, sodium methyl stearoyl taurate, and combinations of two or more thereof. In some embodiments, the oral care composition comprises sodium lauroyl methyl taurate (or sodium methyl lauroyl taurate), sodium methyl cocoyl taurate (SMCT), or a combination thereof.

The anionic surfactant(s) may have at least one acyl group, preferably, comprising a carbon chain of 8 to 21 carbons. In some instance, the alkyl group(s) of the anionic surfactant(s) comprise a carbon chain of 8 to 19 carbons, 8 to 17 carbons, 8 to 15 carbons, 8 to 13 carbons, 8 to 11 carbons; 9 to 21 carbons, 9 to 19 carbons, 9 to 17 carbons, 9 to 15 carbons, 9 to 13 carbons, 9 to 11 carbons; 11 to 21 carbons, 11 to 19 carbons, 11 to 17 carbons, 11 to 15 carbons, 11 to 13 carbons; 13 to 21 carbons, 13 to 19 carbons, 13 to 17 carbons, or any range or subrange thereof. The anionic surfactants disclosed herein may be incorporated in a salt form. The salt form of the anionic surfactants may have an alkali metal (e.g., sodium or potassium) and/or ammonium group.

The oral care composition may include, or in some embodiments exclude, one or more amphoteric surfactants, such as those having a substituent containing 8 to 18 carbon atoms and a substituent containing one or more carboxylate, sulfonate, sulfate, phosphate, or phosphonate. Examples of amphoteric surfactants include those having an alkyl group comprising from 8 to 20 carbon atoms, 8 to 16 carbon atoms, 10 to 16 carbon atoms, or 10 to 13 carbon atoms. The amphoteric surfactant(s) may include, but are not limited to, derivatives of aliphatic secondary and tertiary amines in which the aliphatic radical can be straight chain or branched. In some cases, one of the aliphatic substituents of the amphoteric surfactant contains about 8 to about 18 carbon atoms and one of the aliphatic substituents contains an anionic water solubilizing group, e.g., carboxy, sulfonate, sulfate, phosphate, or phosphonate. The amphoteric surfactants disclosed herein may be present in the oral care film in a salt form.

The amphoteric surfactants may be selected from alkyl amphopropionates, betaines, alkyl sultaines, alkyl amphoacetates, or a combination of two or more thereof. In some cases, the amphoteric surfactant is selected from betaine surfactants. Examples of betaine surfactants include, e.g., alkyl betaines, such as coco dimethyl carboxymethyl betaine, lauryl dimethyl carboxy-methyl betaine, lauryl dimethyl alpha-carboxyethyl betaine, cetyl dimethyl carboxymethyl betaine, lauryl bis-(2-hydroxyethyl)carboxy methyl betaine, stearyl bis-(2-hydroxypropyl)carboxymethyl betaine, oleyl dimethyl gamma-carboxypropyl betaine, lauryl bis-(2-hydroxypropyl)alpha-carboxyethyl betaine. In some preferred embodiments, the oral care composition may be substantially free of or free of amphoteric surfactants.

Additionally or alternatively, the oral care composition may comprise one or more nonionic surfactant(s). The oral care composition may include nonionic surfactant(s) in an amount of about 0.5 to about 10 wt. %, based on the total weight of the oral care composition. In some instances, the nonionic surfactant(s) is present in the oral care composition in an amount from about 0.5 to about 10 wt. %, about 0.5 to about 8 wt. %, about 0.5 to about 6 wt. %, about 0.5 to about 5 wt. %, about 0.5 to about 4 wt. %, about 0.5 to about 3 wt. %, about 0.5 to about 2 wt. %; from about 1 to about 10 wt. %, about 1 to about 8 wt. %, about 1 to about 6 wt. %, about 1 to about 5 wt. %, about 1 to about 4 wt. %, about 1 to about 3 wt. %, about 1 to about 2 wt. %; from about 2 to about 10 wt. %, about 2 to about 8 wt. %, about 2 to about 6 wt. %, about 2 to about 5 wt. %, about 2 to about 4 wt. %, about 2 to about 3 wt. %; from about 3 to about 10 wt. %, about 3 to about 8 wt. %, about 3 to about 6 wt. %, about 3 to about 5 wt. %, about 3 to about 4 wt. %; from about 5 to about 10 wt. %, about 5 to about 8 wt. %, about 5 to about 6 wt. %; from about 7 to about 10 wt. %, about 7 to about 8 wt. %, about 8 to about 10 wt. %, or any range or subrange thereof, based on the total weight of the oral care composition.

The one or more nonionic surfactant(s) may be selected from glucosides, compounds produced by the condensation of alkylene oxide groups (hydrophilic in nature) with an organic hydrophobic compound, which may be aliphatic or alkylaromatic in nature. Examples of glucoside surfactants include decyl glucoside, stearyl glucoside, lauryl glucoside, coco-glucoside, cetearyl glucoside, decyl lauryl glucoside, lauroyl ethyl glucoside, myristoyl ethyl glucoside, oleoyl ethyl glucoside, or a combination of two or more thereof. In some embodiments, the surfactant system includes one or more nonionic surfactant(s) chosen from lauryl glucoside, lauroyl ethyl glucoside, myristoyl ethyl glucoside, oleoyl ethyl glucoside, and a combination of two or more thereof. Additionally or alternatively, the glucoside surfactant may be chosen from polyglucosides, such as alkylpolyglucosides.

Further examples of nonionic surfactants include poloxamers, polyethylene oxide condensates of alkyl phenols, products derived from the condensation of ethylene oxide with the reaction product of propylene oxide and ethylene diamine, ethylene oxide condensates of aliphatic alcohols, acids, and esters, long chain tertiary amine oxides, long chain tertiary phosphine oxides, long chain dialkyl sulfoxides and mixtures of such materials. Additional examples of nonionic surfactants include polyoxyethylene, polyoxyethylene sorbitan esters, polyoxyl n40-60 hydrogenated castor oil, fatty alcohol ethoxylates, polyethylene oxide condensates of alkyl phenols, products derived from the condensation of ethylene oxide with the reaction product of propylene oxide and ethylene diamine, ethylene oxide condensates of aliphatic alcohols, long chain tertiary amine oxides, long chain tertiary phosphine oxides, long chain dialkyl sulfoxides, or a combination of two or more thereof. In some instances, the nonionic surfactant comprises amine oxides, fatty acid amides, ethoxylated fatty alcohols, block copolymers of polyethylene glycol and polypropylene glycol, glycerol alkyl esters, polyoxyethytene glycol octylphenol ethers, sorbitan alkyl esters, polyoxyethylene glycol sorbitan alkyl esters, or a combination of two or more thereof. In some preferred embodiments, the oral care composition may be substantially free of or free of nonionic surfactants.

The oral care compositions may include an anti-calculus agent. The amount of anti-calculus agent(s) present in the oral are composition may be from about 0.5 to about 10 wt. %, based on the total weight of the oral care composition. In some instances, the anti-calculus agent(s) is present in the oral care composition in an amount from about 0.5 to about 10 wt. %, about 0.5 to about 8 wt. %, about 0.5 to about 6 wt. %, about 0.5 to about 5 wt. %, about 0.5 to about 4 wt. %, about 0.5 to about 3 wt. %, about 0.5 to about 2 wt. %; from about 1 to about 10 wt. %, about 1 to about 8 wt. %, about 1 to about 6 wt. %, about 1 to about 5 wt. %, about 1 to about 4 wt. %, about 1 to about 3 wt. %, about 1 to about 2 wt. %; from about 2 to about 10 wt. %, about 2 to about 8 wt. %, about 2 to about 6 wt. %, about 2 to about 5 wt. %, about 2 to about 4 wt. %, about 2 to about 3 wt. %; from about 3 to about 10 wt. %, about 3 to about 8 wt. %, about 3 to about 6 wt. %, about 3 to about 5 wt. %, about 3 to about 4 wt. %; from about 5 to about 10 wt. %, about 5 to about 8 wt. %, about 5 to about 6 wt. %; from about 7 to about 10 wt. %, about 7 to about 8 wt. %, about 8 to about 10 wt. %, or any range or subrange thereof, based on the total weight of the oral care composition.

The anti-calculus agent(s) may be chosen from phosphates and polyphosphates (e.g., pyrophosphates), polyaminopropanesulfonic acid (AMPS), hexametaphosphate salts, zinc citrate trihydrate, polypeptides, polyolefin sulfonates, polyolefin phosphates, diphosphonates, and combinations of two or more thereof. Examples of anti-calculus agents include tetrasodium pyrophosphate, ammonium tripolyphosphate, hexametaphosphate salt, polycarboxylate polymers, polyvinyl methyl ether/maleic anhydride (PVME/MA) copolymers, or combinations of two or more thereof. In some embodiments, the oral care composition includes one or more alkali metal polyphosphate, e.g., as tetrasodium pyrophosphate.

In some embodiments, the oral care compositions may include one or more sweeteners. The oral care composition may include caloric sweeteners and/or non-caloric sweeteners. Examples of non-caloric sweeteners include saccharin, e.g., sodium saccharin, acesulfame, neotame, cyclamate or sucralose; natural high-intensity sweeteners, such as thaumatin, stevioside or glycyrrhizin; or sugar alcohols, such as sorbitol, xylitol, maltitol and mannitol. Examples of caloric sweeteners include sugars, such as fructose, glucose, sucrose, and high fructose syrups.

The one or more sweetener(s) may be present in the oral care composition in an amount from about 0.1 to about 7 wt. %, based on the total weight of the oral care composition. For example, the oral care composition may have a total amount of sweetener(s) from about 0.1 to about 5 wt. %, about 0.1 to about 4 wt. %, about 0.1 to about 3 wt. %, about 0.1 to about 2 wt. %, about 0.1 to about 1 wt. %; from about 0.5 to about 7 wt. %, about 0.5 to about 5 wt. %, about 0.5 to about 4 wt. %, about 0.5 to about 3 wt. %, about 0.5 to about 2 wt. %, about 0.5 to about 1 wt. %; from about 1 to about 7 wt. %, about 1 to about 5 wt. %, about 1 to about 4 wt. %, about 1 to about 3 wt. %, about 1 to about 2 wt. %; from about 2 to about 7 wt. %, about 2 to about 5 wt. %, about 2 to about 4 wt. %, about 2 to about 3 wt. %; from about 3 to about 7 wt. %, about 3 to about 5 wt. %, about 3 to about 4 wt. %; from about 4 to about 7 wt. %, about 4 to about 5 wt. %; from about 5 to about 7 wt. %, or any range or subrange thereof, based on the total weight of the oral care composition.

In some embodiments, the oral care composition preferably is substantially free or free of caloric sweeteners. For example, the oral care compositions may have about 4 wt. % or less, about 3 wt. % or less, about 2 wt. % or less, about 1 wt. % or less, about 0.5 wt. % or less, or about 0.1 wt. % or less, based on the weight of the oral care composition. In at least one embodiment, the oral care composition contains about 0 wt. % or 0 wt. % of caloric sweeteners, based on the weight of the oral care composition.

The oral care compositions of the present disclosure may include a flavoring agent. The flavoring agent is typically incorporated in the oral care composition at a concentration of about 0.01 to about 3 wt. % by weight of the oral care composition. For example, the amount of flavoring agent(s) present in the oral care composition may be from about 0.01 to about 2 wt. %, about 0.01 to about 1 wt. %, about 0.01 to about 0.5 wt. %, about 0.01 to about 0.1 wt. %; from about 0.1 to about 3 wt. %, about 0.1 to about 2 wt. %, about 0.1 to about 1 wt. %, about 0.1 to about 0.5 wt. %; from about 0.5 to about 3 wt. %, about 0.5 to about 2 wt. %, about 0.5 to about 1 wt. %; from about 1 to about 3 wt. %, about 1 to about 2 wt. %; from about 2 to about 3 wt. %, including any range or subrange thereof, based on the total weight of the oral care composition.

Suitable flavoring agents include, but are not limited to, essential oils and various flavoring aldehydes, esters, alcohols, and similar materials. Examples of the essential oils include oils of spearmint, peppermint, wintergreen, sassafras, clove, sage, eucalyptus, marjoram, cinnamon, lemon, lime, grapefruit, and orange. Also useful are such chemicals as menthol, carvone, and anethole. Additional flavoring agents may include, but are not limited to menthol, artificial vanilla, cinnamon derivatives, various fruit flavors, spearmint oil, peppermint oil, cinnamon oil, oil of wintergreen (methylsalicylate), clove oil, bay oil, anise oil, eucalyptus oil, thyme oil, cedar leaf oil, oil of nutmeg, oil of sage, oil of bitter almonds, cassia oil, and a combination of two or more thereof.

The oral care compositions may include one or more colorants. Exemplary colorants can include natural or uncertified colors from natural sources or certified colors for the effect of color. In some embodiments, the colorant can include dyes, certified aluminum lakes or colors derived from a natural source. The colorant may be water-based, oil-based or dry. The colorants can be primary colors, blends of colors or discrete mixtures of colors, such as confetti. The concentrations of the colorant in the oral care composition may be from trace amount to about 0.6 wt. %, from about 0.1 to about 0.5 wt. %, about 0.2 to about 0.4 wt. %, or about 0.15 to about 0.35 wt. %, based on the total weight of the oral care composition.

The oral care composition may include one or more pH adjusters to increase or decrease the overall pH of the oral care composition. For example, one or more acids may be included to decrease the pH of the oral care composition. Examples of suitable acids for decreasing the pH of the oral care composition include, but are not limited to, citric acid, acetic acid, and the like. The oral care composition may include one or more bases, such as sodium hydroxide, potassium hydroxide and the like, to increase the pH of the oral care composition. Additional or alternative acids and bases that are suitable for adjusting the pH of the oral care composition are readily known to one of ordinary skill in the art.

The amount of the pH adjuster in the oral care composition may be based on the desired pH of the final oral care composition and/or product. For example, the total amount of the pH adjuster may range from about 0.05 to about 20 wt. %, based on the total weight of the oral care composition. In some instances, the total amount of pH adjuster is from about 0.05 to about 15 wt. %, about 0.1 to about 10 wt. %, or about 0.12 to about 5 wt. %, including ranges and sub-ranges thereof, based on the total weight of the oral care composition.

In another aspect, the present disclosure provides a method of using the oral care compositions disclosed herein. The methods typically comprise applying any of the compositions as described herein to the teeth, e.g., by brushing, rubbing, swishing when the oral care composition is in the form of a dentifrice, or otherwise administering the oral care compositions to the oral cavity of a subject. The oral care compositions can be administered regularly, such as, for example, one or more times per day.

### EXAMPLES

### Example 1

Two non-limiting, example oral care compositions (Example Compositions A and B) were prepared in the form of dentifrices in accordance with aspects of the invention. A comparative oral care composition (Comparative Composition 1) was also prepared in the form of a dentifrice. The amount of sodium acid pyrophosphate was different between Example Composition A, Example Composition B, and Comparative Composition 1 to maintain similar pH levels. The formulation for Example Compositions A and B and Comparative Composition 1 is provided in Table 1.

TABLE 1

| | Comp. 1 | Ex. A | Ex. B |
|---|---|---|---|
| US INCI Name | (wt. %) | (wt. %) | (wt. %) |
| Hydrogen Peroxide | 4.2 | 4.2 | 4.2 |
| Polyvinyl pyrrolidone | 9 | 8.5 | 8.8 |
| Carbomer 974P | | 1 | 0.5 |
| Fumed Silica | 3.4 | 3.4 | 3.4 |
| Calcium Pyrophosphate | 15 | 15 | 15 |
| Tetrasodium Pyrophosphate | 1.3 | 1.8 | 1.5 |
| Sodium Acid Pyrophosphate | 0.5 | | 0.3 |
| Sodium Lauryl Sulfate | 2 | 2 | 2 |
| Sodium Monofluorophosphate | 0.8 | 0.8 | 0.8 |
| Flavor | 2.3 | 2.3 | 2.3 |
| Sodium Saccharin and Sucralose | 0.9 | 0.9 | 0.9 |
| Preservatives (e.g., Butylated | 0.01-0.2 | 0.01-0.2 | 0.01-0.2 |
| Hydroxytoluene) | | | |
| Water | 4.2 | 4.2 | 4.2 |
| Polyethylene Glycol/Polypropylene | 7.5 | 7.5 | 7.5 |
| Glycol 116/66 Copolymer | | | |
| Propylene Glycol | Q.S. | Q.S. | Q.S. |
| | to 100 | to 100 | to 100 |

### Example 2

Example Compositions A and B were evaluated in comparison to Comparative Composition 1 using HAP disks to assess the stain resistance of Example Compositions A and B. To determine the initial color of the HAP disks, each of the HAP disks was placed in well plates and soaked in 4 mL of water and then incubated for an hour. The HAP disks were removed from the well plates, pat dried, and evaluated using a SpectraShade device to determine the L, a, and b values of each disk. L* represents lightness from black to white on a scale of zero to 100, while a* and b* represent chromaticity with no specific numeric limits. Negative a* corresponds with green, positive a* corresponds with red, negative b* corresponds with blue and positive b* corresponds with yellow.

A dentifrice slurry sample was then applied to the HAP disks. The dentifrice slurry samples were prepared by combining a sample of either Example Composition A, Example Composition B, or Comparative Composition 1 with water in amounts to produce dentifrice slurry samples having a weight ratio of the respective dentifrice to water of 1:2. The HAP disks were then placed in separate well plates and 4 mL of respective dentifrice slurry samples was added thereto for 2 minutes. The HAP disks were then removed from the well plates and rinsed with 5 mL of water three times.

The HAP disks were then stained with a coffee solution, which was prepared by dissolving 2 grams of coffee for every 98 grams of water. Specifically, the HAP disks were placed in separate well plates and 4 mL of the coffee solution was added thereto. The HAP disks were kept in the coffee solution for 180 minutes. The HAP disks were then removed from the well plates and rinsed with 5 mL of water three times. The HAP disks were pat dried and then immediately evaluated to assess the final color of the HAP disks using a SpectraShade to determine the final L, a, and b values of the HAP disks. A summary of the change in color or whiteness (AW*) for Composition A, Example Composition B, and Comparative Composition 1 is shown in Table 2 (below).

TABLE 2

| | Comp. 1 | Ex. A | Ex. B |
|---|---|---|---|
| ΔW* (W*_{Initial} − W*_{Final}) | 22.0 ± 2.2 | 15.4 ± 4.3 | 20.9 ± 4.1 |

While the present invention has been described with reference to several embodiments, which embodiments have been set forth in considerable detail for the purposes of making a complete disclosure of the invention, such embodiments are merely exemplary and are not intended to be limiting or represent an exhaustive enumeration of all aspects of the invention. The scope of the invention is to be determined from the claims appended hereto. Further, it will be apparent to those of skill in the art that numerous changes may be made in such details without departing from the spirit and the principles of the invention.

What is claimed is:

1. An oral care composition comprising:
a whitening agent comprising from 0.1 to about 10 wt. % of a peroxide;
a cross-linked polyacrylic acid and/or a salt thereof,
a polyol; and
water in an amount up to 10 wt. %, wherein all weight percentages are based on the total weight of the oral care composition.

2. The oral care composition according to claim 1, wherein the peroxide is present in an amount of about 3 to about 10 wt. %.

3. The oral care composition according to claim 1, wherein the peroxide is selected from hydrogen peroxide, lithium peroxide, potassium peroxide, sodium peroxide, magnesium peroxide, calcium peroxide, barium peroxide, alkyl hydrogen peroxides, dialkyl peroxides, alkyl peroxy acids, peroxy esters, diacyl peroxides, benzoyl peroxide, monoperoxyphthalate, linear PVP complexed with hydrogen peroxide, cross-linked PVP complexed with hydrogen peroxide, silica complexed with hydrogen peroxide, tetrasodium pyrophosphate, complexed with hydrogen peroxide, and a combination of two or more thereof.

4. The oral care composition according to claim 1, wherein the peroxide is a non-complexed peroxide.

5. The oral care composition according to claim 1, wherein the peroxide is hydrogen peroxide.

6. The oral care composition according to claim 1, wherein the cross-linked polyacrylic acid and/or a salt thereof is a branched polyacrylic acid and/or a salt thereof.

7. The oral care composition according to claim 1, wherein the cross-linked polyacrylic acid and/or the salt thereof comprises a carbomer.

8. The oral care composition according to claim 1, wherein the cross-linked polyacrylic acid and/or the salt thereof consist of one or more carbomer.

9. The oral care composition according to claim 1, wherein the cross-linked polyacrylic acid and/or the salt thereof is present in an amount from about 0.2 to about 5 wt. %.

10. The oral care composition according to claim 1 further comprising:
an abrasive in an amount from about 1 to about 35 wt. %.

11. The oral care composition according to claim 10, wherein the abrasive comprises an insoluble phosphate salt selected from calcium pyrophosphate, dicalcium orthophosphate dihydrate, tricalcium phosphate, and calcium polymetaphosphate, and a combination of two or more thereof.

12. The oral care composition according to claim 10, wherein the abrasive consists of one or more calcium-based abrasive.

13. The oral care composition according to claim 1, wherein the polyol is present in an amount from about 30 to about 70 wt. %.

14. The oral care composition according to claim 13, wherein at least one of the plurality of polyols is selected from glycerin, ethylene glycol, propylene glycol, butylene glycol, pentylene glycol, hexylene glycol, dipropylene glycol, a polyethylene glycol, a polypropylene glycol, and a combination of two or more thereof.

15. The oral care composition according to claim 1 further comprising:
a fluoride source in an amount from about 0.1 to about 7 wt. %, wherein the fluoride source is selected from stannous fluoride, sodium fluoride, potassium fluoride, sodium monofluorophosphate, sodium fluorosilicate, ammonium fluorosilicate, amine fluoride, ammonium fluoride, and a combination of two or more thereof.

16. The oral care composition according to claim 1 further comprising an anti-calculus agent in an amount from about 0.5 to about 10 wt. %, wherein the anti-calculus agent is selected from tetrasodium pyrophosphate, ammonium tripolyphosphate, hexametaphosphate salt, polycarboxylate polymers, polyvinyl methyl ether/maleic anhydride (PVME/MA) copolymers, and a combination of two or more thereof.

17. The oral care composition according to claim 1, wherein the oral care composition is free of a peroxidase.

18. The oral care composition according to claim 1, wherein the oral care composition is free of complexed peroxides.

19. An oral care composition comprising:
a whitening agent comprising from 3 to about 8 wt. % of hydrogen peroxide;
a carbomer;
a polyol; and
from about 1 to about 8 wt. % of water, wherein all weight percentages are based on the total weight of the oral care composition.

20. The oral care composition according to claim 1, wherein the oral care composition has a relative dental abrasivity of about 10 to about 250, as determined pursuant to standards DIN EN ISO 11609.
